(19) United States
(12) Patent Application Publication
(10) Pub. No.: US 2003/0186848 A1
(43) Pub. Date: Oct. 2, 2003

(54) Recombinant iron uptake proteins

(76) Inventors: Andrew Richard Gorringe, Salisbury (GB); Michael John Hudson, Salisbury Wiltshire (GB); Mary Anne Matheson, Camr Porton Down (GB); Andrew Robinson, Salisbury Wiltshire (GB); David McKay West, Salisbury Wiltshire (GB)

Correspondence Address: Leslie Meyer Leon, IP Legal Strategies Group, 1480 Falmounth Road, PO Box 1210, Centerville, MA, 02632-1210, US

(21) Appl. No.: 10/240,218

(22) PCT Filed: Mar. 27, 2001
(86) PCT No.: PCT/GB01/01348

(51) Int. Cl. A61K 38/40; C12P 21/02; C12N 1/21; C07K 14/22; C07H 21/04; C12N 15/74

(52) U.S. Cl. 514/6; 530/400; 435/69.1; 435/252.3; 435/320.1; 536/23.2

(57) ABSTRACT

Neisserial iron uptake proteins including transferrin binding protein A (TbpA) and B (TbpB) in both full length form and as immunogenic fragments are made recombinantly. A non-neisserial cell expresses a TbpA which is extracted from the cell under mild conditions and retains substantially the antigenicity of native TbpA on its surface. TbpB is also made recombinantly and both TbpA and TbpB are included in vaccine compositions.

The present invention relates to recombinant iron uptake proteins, in particular recombinant transferrin binding proteins and vaccines based thereon.

Meningococcal meningitis is of particular importance as a worldwide health problem and in many countries the incidence of infection is increasing. Neisseria meningitidis (the meningococcus) is the organism that causes the disease and is also responsible for meningococcal septicaemia, which is associated with rapid onset and high mortality, with around 22% of cases proving fatal.

The meningococcal transferrin receptor is a suitable vaccine component and made up of two types of component protein chain, transferrin binding protein A (TbpA) and TbpB. The receptor complex is proposed to be formed from a dimer of TbpA which associates with a single TbpB. Epitopes present in TbpA are known to be masked within the interior of the protein. Vaccines against meningococcal disease based on TbpB from one strain alone show some cross reactivity and there is evidence of a cross-reactive immune response in rabbits immunised with TbpB alone.

Obtaining Tbps from natural bacterial sources carries the difficulty of obtaining adequate quantities for industrial vaccine production, together with the near impossibility of culturing large volumes of meningococci under safe conditions. It would therefore be desirable to produce the Tbps recombinantly.

Recombinant production of TbpB is known, providing large amounts of the recombinant protein, extracted from inclusion bodies using conventional techniques.

TbpA is one of a family of proteins referred to as TonB-dependent outer membrane receptors, due to their physical location in the Neisserial membrane and their functional interaction with TonB. TbpB is not TonB-dependent but is added to this group for the purpose of the present application due to its interaction with TbpA in forming the transferrin receptor. This group thus includes Tbps and lactoferrin binding proteins and will be referred to as a whole as the iron uptake proteins.

A difficulty in known methods of recombinantly producing iron uptake proteins is that the resultant proteins are recovered in non-native conformations, undesirable in vaccines based thereon. Another problem is that some iron uptake proteins can not hitherto be made recombinantly, and this is notably the case for TbpA.

Recombinant production of TbpB in E. coli is known from Legrain et al, “Production of lipidated meningococcal transferrin binding protein 2 in Escherichia coli”, Protein Expression and Purification 6, 570-578 (1995). This describes an expression system for production of TbpB and fermenter cultures but does not describe TbpB purification. The TbpB is said to be located in the insoluble fraction of the cellular extracts, indicating that the protein is in an insoluble form, i.e. in inclusion bodies.

Lissolo et al, in Infection and Immunity, 63:884-90 (1995) describes purification of TbpB from the meningococcus using denaturing conditions.

Renauld-Mongenie et al, in J Bacteriol., 179:6400-7 (1997) describes expression of TbpB as maltose binding protein-TbpB fusions, and then purification in 3M Urea buffer using the affinity system for maltose binding proteins.

Gonzalez et al, in Microbiology, 141:2405-16 (1995) describes how Actinobacillus pleuropneumoniae TbpA and TbpB are eluted from transferrin Sepharose with a denaturing buffer containing SDS and mercaptoethanol.

Palmer et al, in FEMS Microbiology Letters, volume 110, pp 139-146, 1993, purport to describe TbpA expression in E. coli. However, Palmer et al were not able to recover TbpA in a functional form.

The invention has as an object the provision of improved methods of production of iron uptake proteins, in particular transferrin binding proteins, and specific objects of providing alternative and/or improved recombinant production of such proteins and of providing recombinant production of TbpA. A further object of the invention is to provide improved preparation of vaccines containing transferrin binding proteins.

A first aspect of the invention is based upon successful recombinant expression of transferrin binding proteins and confirmation that the transferrin binding proteins expressed retain the antigenicity of native transferrin binding proteins, as evidenced by ability of the recombinantly produced proteins to bind human transferrin and confer protective immunity against challenge by meningococci.

Accordingly, the invention provides a non-neisserial cell expressing a neisserial iron uptake protein, wherein the iron uptake protein can be extracted from the cell under mild conditions and retains substantially the antigenicity of native iron uptake protein.

The invention further provides a non-neisserial cell expressing a neisserial iron uptake protein wherein the iron uptake protein is located in a surface membrane of the cell.

In a specific embodiment, the invention provides a non-neisserial cell expressing a Neisserial transferrin binding protein (Tbp) wherein said Tbp can be extracted from the cell under mild conditions and retains substantially the antigenicity of native TbpA.

The invention additionally provides a cell overexpressing a neisserial iron uptake protein.

The iron uptake protein is preferably selected from the group consisting of transferrin binding proteins (Tbps), lactoferrin binding proteins (Lbps), haemoglobin binding protein, enterobactin binding protein, vibriobactin binding protein, ferric siderophore binding protein, heme binding protein, hemin binding protein, chrysobactin binding protein, hydroxymate binding protein and pseudobactin binding protein—some of these proteins also being referred to as “receptor”s rather than “binding protein”s.

Also provided is a cell which expresses a neisserial Tbp, wherein the yield of said Tbp is at least 4 mg per litre of culture, preferably at least 7 mg and more preferably at least 10 mg. In these embodiments of the invention, the cell is preferably bacterial and the Tbp can be A or B.

TbpA is expressed in an example at a yield of from about 6 to about 12 mg per litre of culture.

In addition, the invention is hence of application to overexpression of Tbps in organisms that are known to express Tbps. In this sense, “overexpression” is intended to mean expression in a cell of a protein that is not in nature expressed in that cell as well as expression in a cell of a protein that is expressed in that cell in nature but at a lower level, the invention in this latter case resulting in expression of that protein at a higher level. Overexpression in, for example, commensal neisseria is of use as outer membrane preparations enriched in Tbps or containing heterologous Tbps can be obtained therefrom, and are advantageously used in vaccines. A specific embodiment lies in a commensal Neisseria expressing an iron uptake protein from a pathogenic Neisseria, especially one expressing TbpA and/or TbpB.

In use of the invention, TbpA has been found to be expressed so that it is located on or associated with the cell surface, and thus expressed with any necessary trafficking signals so that the TbpA gene product ends up on or associated with the surface. Further, the TbpA expressed can easily be extracted using mild conditions, such as using a conventional detergent extraction method, whilst retaining the antigenicity and hence the properties relevant to vaccinating use of native protein.

By mild conditions it is meant that recombinant protein can be extracted without the need to denature and then renature the protein. If the protein were located in inclusion bodies there would be a need to employ more severe recovery techniques, typically involving denaturing of the protein. However, an advantage of the invention is that the protein is surface bound or associated, and is not sequestered in inclusion bodies, so extraction does not require this denaturing, with its consequent damage to the confirmation of the protein and potential loss of key epitopes.

It is an option also to produce TbpB recombinantly, and thus the invention also provides a cell expressing, recombinantly, both TbpA and TbpB. This confers the advantage of using a single cell for two important vaccine components.

In an example below, TbpB has been expressed at a yield of from about 7 to about 32 mg per litre of culture.

The desired antigenicity in, by way of example, the TbpA and TbpB obtained from cells of the invention is antigenicity that stimulates an immune response against Tbp and organisms expressing Tbp. The proteins obtained in examples of the invention have been tested in animal models and the extracted Tbp demonstrated to confer protection against subsequent challenge by meningococci, this confirming Tbp in a substantially native confirmation has been obtained. This has the advantage of providing an efficient route of recombinant production of these proteins for subsequent use for example in further structure-function analysis and pharmaceutical, particularly vaccine, applications.

The invention also relates to methods of iron uptake protein production and provides, in a second aspect, a method of producing an iron uptake protein by expressing a recombinant iron uptake protein gene in a non-neisserial cell host such that the protein is expressed and translocated to a surface membrane of the host. The protein can then be extracted using mild conditions.

The invention also relates specifically to methods of Tbp production and provides a method of producing a neisserial transferrin binding protein (Tbp) comprising:

a. expressing a recombinant neisserial Tbp gene in a non-neisserial host such that Tbp protein is expressed and translocated to the cell membrane;

b. under mild conditions, extracting Tbp protein.

The method can also include expressing recombinant neisserial TbpA and TbpB genes, in the same culture and optionally in the same cell.

A still further embodiment of the invention resides in a method of producing a transferrin binding protein (Tbp) from a pathogenic Neisseria, comprising expressing a gene encoding the Tbp in a commensal Neisserial host such that Tbp protein is translocated to an outer surface membrane of the commensal host, extracting the Tbp under mild conditions, and, optionally, purifying said Tbp protein. An outer membrane vesicle preparation is suitable, for example for vaccine preparation, and a N. meningitidis gene expressed in N. lactamica is particularly suitable.

The Tbp is suitably extracted by solubilising membrane associated Tbp in a non-ionic detergent solution, yielding good quantities of Tbp in native form and which has been demonstrated to be both functional and protective against meningococcal challenge. A number of non-ionic detergents are suitable for the extraction, including one chosen from an alkyl glucoside; n-octyl-β-D-glucopyranoside; TRITON® X100; ELUGENT®; dodecyl-maltoside; and n-octyl-β-D-maltoside. The extraction preferably includes a low energy homogenisation step, conveniently preceded by using apparatus such as a bead-beating apparatus, though other such apparatus are also available, to break up cells and isolate cell membranes.

To obtain the desired location of expressed protein, an expression construct is preferably used that combines a nucleotide sequence encoding the iron uptake protein with a leader sequence directing the expressed protein to a surface membrane of the host, this construct forming a further embodiment of the invention. The leader sequence is suitably a neisserial leader sequence, and good results have been obtained in specific examples below where the neisserial iron uptake protein is expressed using its own neisserial leader sequence. TbpA has been expressed using the TbpA leader. Another option is to use a host leader sequence that directs translocation of the recombinant product to a surface membrane of the host, for example an E. coli leader if the protein is made in E. coli. TbpB has been expressed using a host leader.

Once protein has been obtained from the cells expressing the protein it is preferred to subject the crude product obtained to one or more purification processes. These processes may remove such contaminants as other proteins from the host cell, non-proteinaceous contaminants and also other components of the cell. The crude product can be purified by affinity chromatography, and preferably in the case of Tbps using a transferrin-bound affinity matrix. In this respect, reference to transferrin encompasses fragments, variants and derivatives of human transferrin that retain transferrin's binding to Tbps. A further aspect of the invention, described in more detail below, relates to recombinant transferrin, and the affinity matrix preferably comprises recombinant human transferrin.

A third aspect of the invention lies in a method of preparing a vaccine, comprising obtaining TbpA, TbpB or TbpA and TbpB according to the invention and combining said Tbp with a pharmaceutically acceptable carrier. The invention further provides use of a cell according to the invention in manufacture of Tbp, and use of a cell according to the invention in manufacture of a vaccine for protection against neisserial disease and/or meningococcal disease.

As described in examples below in more detail, transferrin binding proteins have been expressed recombinantly in E. coli. The invention is nevertheless of application to a range of host cell types, both prokaryotic and eukaryotic, such as yeast (eg. Saccharomyces cerevisiae, Pichia pastoris), insect cells (eg. baculovirus expression system), gram positive bacterial expression systems (eg. Bacillus subtilis) and mammalian cell culture. The expression vectors used in the invention have been designed for use in E. coli and corresponding vectors can be designed for use in other bacterial hosts, subject to selection of suitable promoters and origins of replication according to the host cell chosen. Examples of suitable cloning techniques and other hosts are described, for example, in Sambrook et al “Molecular Cloning: A laboratory Manual”, 1989.

The iron uptake proteins expressed according to the invention are derived from Neisseria. In the specific embodiments, transferrin binding proteins from pathogenic Neisseria, specifically N. meningitidis, have been expressed, more specifically of strain K454. Other neisserial transferrin binding proteins may suitably be expressed according to the invention, whether from virulent or a virulent strains and also from commensal strains. By reference to “iron uptake proteins” and “transferrin binding protein” it is intended to include whole, intact protein and also fragments and derivatives and variants thereof, provided that said fragments, derivatives and variants when administered in a vaccinating composition confer protection against subsequent challenge by meningococci and/or gonococci.

It is a significant advantage of the invention that it is now possible to produce iron uptake proteins and especially transferrin binding proteins in conformation suitable for vaccines and in useful quantities using the recombinant techniques of the invention.

A fourth aspect of the invention provides a method of purifying a Tbp-containing preparation, comprising eluting the preparation through an affinity matrix comprising immobilized transferrin.

A benefit of this method is that the affinity matrix will only bind functional transferrin binding protein, as only functional protein will bind to the immobilised transferrin. In this respect, reference to transferrin encompasses fragments, variants and derivatives of transferrin that retain transferrin's binding to Tbps. Thus, the eluate is both purified in respect of proteins that are not transferrin binding proteins and is also purified in that transferrin binding protein which is non-functional, mutated or otherwise does not bind transferrin passes through the matrix. It is preferred that human transferrin binding protein be immobilised, more preferably recombinant human transferrin binding protein, which confers a particular benefit in that the purified Tbp and hence preparation of a vaccine for human use is simplified as exclusive processes for removal of these contaminants can be avoided.

A further aspect of the invention provides an affinity matrix for purification of Tbps comprising human recombinant transferrin or a fragment thereof that binds to Tbp. The transferrin may be produced according to a method of the invention described below.

Typically, Tbp is expressed in a host cell and an extract of membrane-bound or membrane-associated Tbp obtained, such as using the mild extraction conditions described above. This extract, a crude Tbp-containing extract, is passed through the matrix, Tbp that can bind immobilized transferrin or fragment thereof is retained whilst non-functional Tbp and other contaminants pass through. The purified Tbp can then be separated from the matrix using conventional techniques, such as low pH.

A further composition of the invention contains a Tbp, wherein at least 90 percent by weight of said Tbp is active Tbp. The Tbp can be A or B, and by active is meant that the Tbp binds to transferrin. The composition is preferably free of Tbp that is not capable of binding transferrin.

For purification of Tbps, recombinant human transferrin can be obtained by:

A. obtaining a clone of human transferrin, or a fragment or derivative thereof;

B. inserting said clone, or fragment or derivative thereof, into an expression vector;

C. expressing the vector in a suitable host organism; and

D. isolating the expressed gene product from said host organism.

The clone can be isolated via a PCR based method, and the expression vector can be selected from the group consisting of pMTL and pET. The host organism is typically a bacterium, suitably E. coli, and specific embodiments of the invention use a host selected from the group consisting of Novablue DE3; HMS 174 DE3; BL21 DE3; JM 109; RV 308; and XL1 Blue.

The invention is now described in specific embodiments, illustrated by the accompanying drawings in which:—

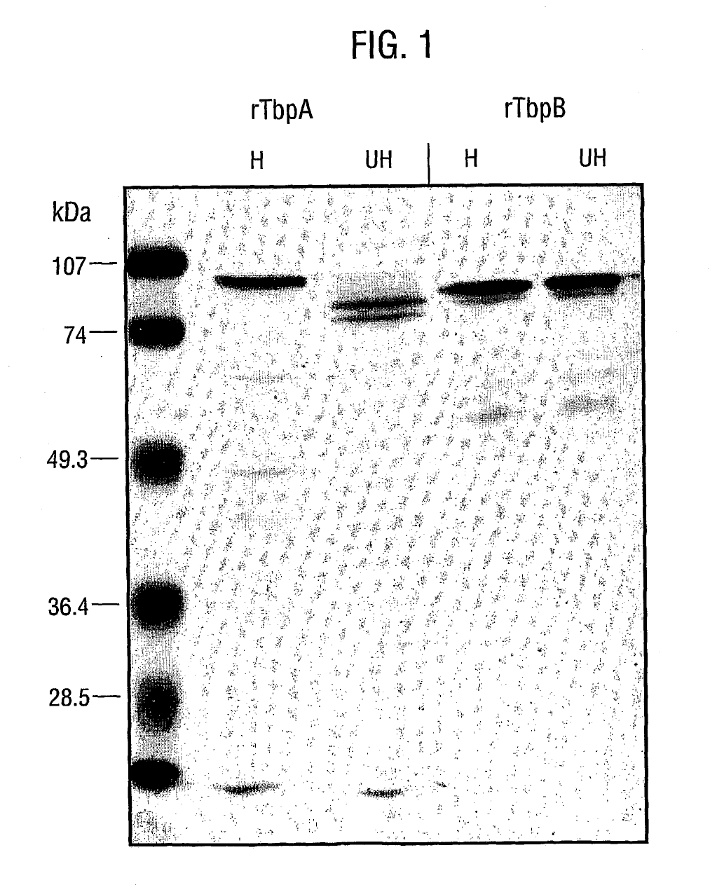

FIG. 1 shows analysis of purified rTbps by SDS-PAGE (10%) under different denaturing conditions;

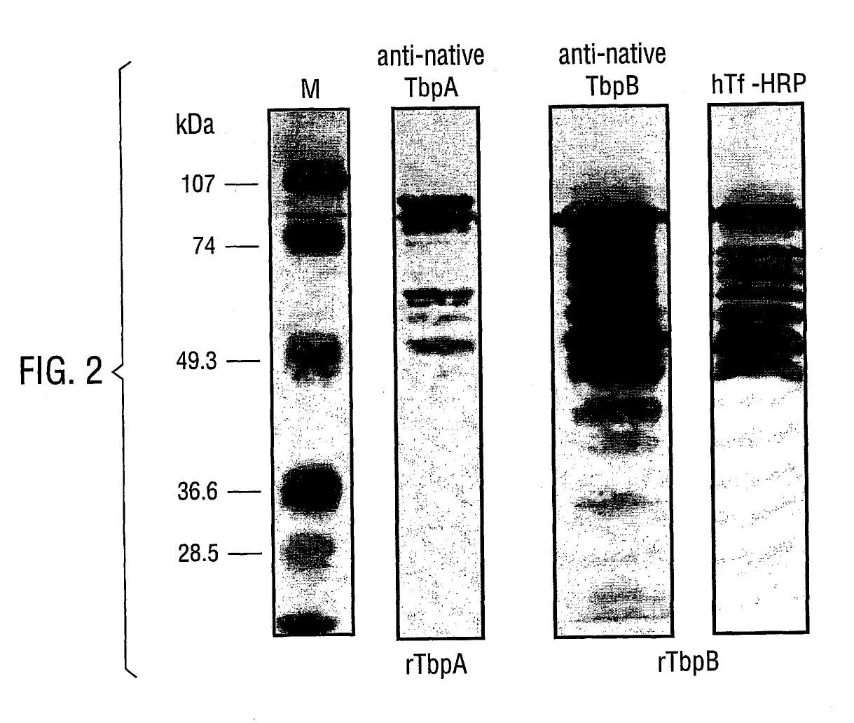

FIG. 2 shows purified rTbps electrophoretically transferred to nitrocellulose membrane and probed with antibodies raised against native Tbps and hTf-HRP conjugate;

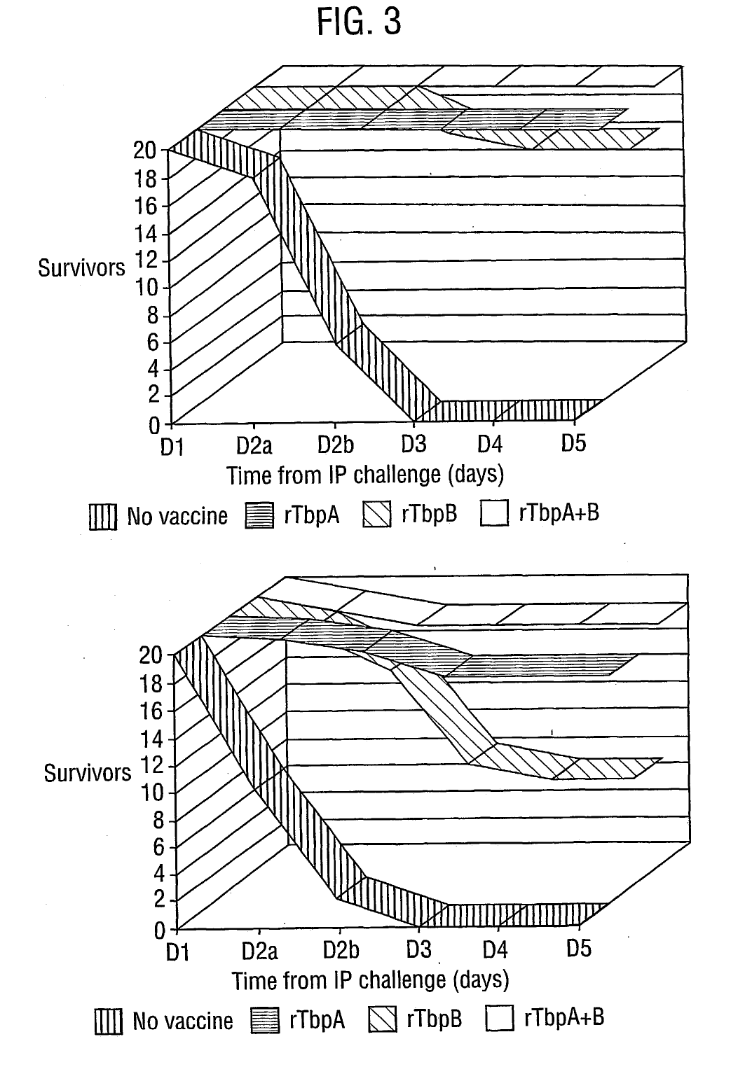

FIG. 3 shows protection against IP challenge with N. meningitidis strain K454 conferred by rTbpA and rTbpB; and

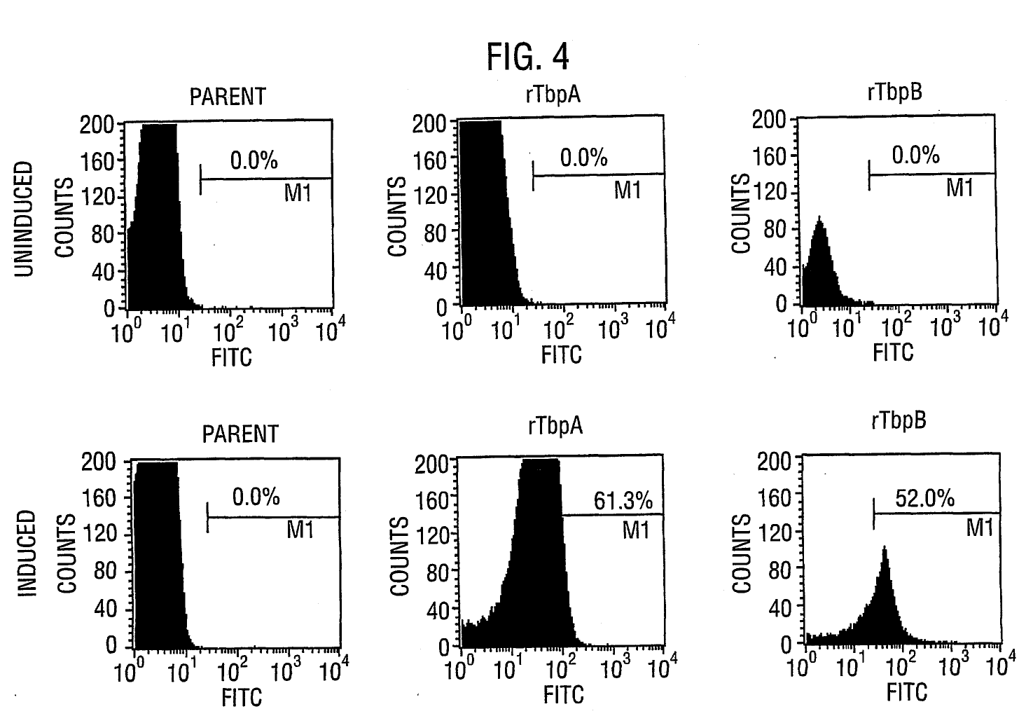

FIG. 4 shows flow cytometry analysis indicating the surface location of expressed Tbps.

In more detail, for FIG. 1, a 10% SDS-PAGE gel of rTbps was stained with Gelcode blue. M is Pharmacia low range molecular weight markers, H is rTbp boiled at 100 degrees C. for 5 minutes before loading onto the gel, UH is rTbp not heated before loading onto the gel. For FIG. 2, rTbps were run on 10% SDS-PAGE gels and Western blotted onto nitrocellulose membrane. M is Biorad prestained molecular weight markers. A consistent pattern of degradation products is seen for batches of rTbpA and rTbpB when probed with serum to the native protein. For rTbpA the same pattern of bands is also seen when human transferrin HRP is used to probe the membrane. In FIG. 3, the number of survivors per group of 20 mice is shown against days from intraperitoneal challenge by N. meningitidis strain K454. The challenge dose for the upper panel was 5×10⁶ and for the lower was 5×10^{7.} In FIG. 4, surface labelling of E. coli JM109 with HTf-FITC is illustrated as counts shown against FITC.

### EXAMPLE 1

tbp Gene Cloning and Expression

Primers to amplify N. meningitidis strain K454 (B15:P1.7, 16) tbps were designed using information from other sequenced N. meningitidis tbp genes in the Genbank database (and subsequent sequencing of the N and C termini of each tbp gene). 5′ primers engineered an NdeI (CATATG) site at the ATG start codon, and 3′ primers contained a BamHI (tbpA) or EcoRI (tbpB) site after the stop codon.

5′tbpA TTAGGGAAACCATATGCAACAGCAAC
3′tbpA GACGGATCCGCGTTTGGACGTTTAAAACTTC
5′tbpB GAATTGGATTTCATATGAACAATCC
3′tbpB GACGAATTCCGGCAGCCGTGCTTATCGC

Restriction Sites in Bold.

tbpB was further modified by replacing the native TbpB leader peptide coding sequence with that of the E. coli RlpB lipoprotein. Genes were cloned into pET22b (Novagen) initially, on an NdeI-BamHI (tbpA) or NdeI-EcoRI (rlpB::tbpB fragment and both strands of each clone sequenced to confirm gene integrity. These same fragments were subsequently subcloned into various pMTL vectors from which optimum expression was found in pMTL2010 (incorporating a lac UV5 promoter driving expression and a tetracycline resistance gene). Plasmid constructs were used to transform a variety of DE3 lysogens for pET22b directed expression (E. coli Novablue DE3, HMS 174 DE3 and BL21 DE3). pMTL2010 clones were used to transform E. coli JM109 and RV308 strains for expression.

We succeeded in expressing TbpA from all vectors, except from pMTL1015 and 2015 under the control of the mdh promoter which is stronger than the lac UV5 promoter. This suggests that strong gene expression causes toxicity. However, the rate of transcription in the pET expression system is greater than would be expected using mdh promoter driven expression, but no studies have been done to address this question. tbpA expressed better in BL21 DE3 than Novablue DE3 when cloned into pET vectors.

Replacement of the native neisserial TbpA leader sequence with that of CPG2 and PeIB leaders still gave rise to active protein (in terms of hTf binding) but offered no improvement over the native neisserial leader.

Sequence data for N. meningitidis strain K454 tbpA are shown below.

Production of Recombinant Meningococcal Tbps in E. coli

1. Growth of Recombinant E. coli Strains

Recombinant E. coli strains (JM109 containing CAMR pMTL vectors with either tbpA or tbpB gene inserted) were grown up in 8 litre fermenters. Soytone-based production medium containing the appropriate antibiotic (either 1.25 mg/l tetracycline or 100 mg/l ampicillin) was used (see table 1). The fermenters were maintained at a temperature of 37° C. with an air flow of 0.5 vessel volumes per minute and a pH of 6.8-7. The dissolved oxygen tension was maintained at >40% by agitation. The cultures were allowed to grow until an A 600 nm of approximately 10 was reached, at which point Tbp expression was induced by the addition of IPTG to a final concentration of 1.0 mM. Cultures were then allowed to grow for a further 6-8 hours. Cells were harvested by centrifugation and the wet weight was determined.

Table 1
Components of Soytone Based medium

| Medium Ingredient | Amount (g/l) | |
|---|---|---|
| Bacto Soytone | 40 | |
| K₂HPO₄ | 4 | |
| KH₂PO₄ | 1 | |
| NH₄Cl | 1 | |
| CaCl₂.2H₂O | 0.01 | |
| K₂SO₄ | 2.6 | |
| Yeast extract | 3 | |
| Glycerol | 41 | |
| Trace elements | 5 | ml |
| 1M MgCl₂.6H₂O | 1 | ml |
| Mazu DF8005 | O.1 | ml |
| Ampicillin/Tetracycline | 100 | mg/1.25 mg |

2. Preparation of Whole Cell Suspension

Recombinant E. coli cells harvested from fermenters were resuspended in 100 mM Tris-HCl buffer, pH 8.0, containing 0.5M NaCl. Cells were resuspended at 10% (w/v) and a hand held glass homogeniser was used to obtain an even suspension. For a typical experiment 40 g of cells were used and resuspended in 400 ml of buffer.

Two methods were used to obtain soluble recombinant transferrin binding proteins (rTbps) for affinity purification from the above cell suspension.

3. Extraction of Tbps

3.1 Procedure A. Direct Extraction From Whole Cells

An equal volume of 100 mM Tris-HCL buffer, pH 8.0, containing 0.5M NaCl and 4% (v/v) Elugent™ detergent (Calbiochem) was added to the whole cells and mixed thoroughly.

The suspension was incubated with gentle stirring at 4° C. for 16 h and then centrifuged at 39000 g for 40 min to remove bacterial debris. The supernatant containing soluble rTbps was gently decanted off in preparation for affinity chromatography.

3.2 Procedure B. Extraction From Membrane Preparations

Crude membranes were prepared by disrupting cells with a bead-beater (Biospec Products, OK, US.). The cell suspension was transferred to a vessel half filled with 0.25-0.5 mm diameter glass beads. The vessel was sealed and placed on to the bead-beating apparatus. The suspension was beaten for 15 seconds to disrupt the cells. Once the beads had settled the suspension was decanted off and centrifuged at 8000 g for 30 min. The supernatant was discarded and the pellet containing crude membranes was resuspended in the original volume of 100 mM Tris-HCl buffer, pH 8.0, containing 0.5M NaCl. Once an even suspension was obtained an equal volume of Tris-HCl buffer, pH 8.0, containing 0.5M NaCl and 4% (v/v) Elugent™ detergent was added. The suspension was incubated with gentle stirring at 4° C. for 16 h. The suspension was then centrifuged at 39000 g for 40 min and the supernatant containing soluble rTbps was decanted off in preparation for affinity chromatography.

4. Preparation of Transferrin-Sepharose Affinity Matrix

Transferrin-Sepharose affinity matrix was prepared using cyanogen bromide (CNBr) activated Sepharose 4B (Pharmacia Biotech) and human transferrin (Sigma). Transferrin purified from human blood will eventually be replaced with recombinant transferrin produced in E. coli. 15 g of CNBR activated Sepharose was suspended in 200 ml of 1 mM HCl and was washed for 15 min with another 2 l of the same solution on a sintered glass filter. 0.369 of human transferrin were dissolved in 50 ml coupling buffer (0.1 M NaHCO₃, pH 8.3, containing 0.5M NaCl) and mixed with the washed Sepharose 4B. The mixture was incubated overnight at 4° C. with gentle mixing. Excess uncoupled transferrin was then washed away with 250 ml of coupling buffer and remaining active groups were blocked by incubating with 0.1 M Tris-HCl buffer, pH 8.0, for 2 h. The transferrin-Sepharose was then washed with 3 cycles of low and high pH buffer using 250 ml of buffer for each wash. The low pH buffer was 0.1M acetate, pH 4.0, and the high pH buffer was 0.1M Tris-HCl, pH 8. Both contained 0.5M NaCl. The transferrin-Sepharose was stored at 4° C. in Tris-HCl, pH 8, until use.

5. Affinity Chromatography

Supernatants containing rTbp A or rTbpB were loaded on to a 10 ml column of human transferrin linked to Sepharose 4B at a flow rate of 1 m/min. For rTbpB the column was saturated with iron by passing 200 ml of iron saturation buffer (40 mM Tris, 2 mM NaHCO₃, 25 mM Na citrate and 1 mM FeSO₄₀.7H₂O) through the column. For rTbpA this step was not necessary. The column was then washed with 20 column volumes of 100 mM Tris-HCl buffer, pH 8.0, containing 0.5M NaCl to remove unbound material. rTbps were eluted from the column using 50 mM glycine buffer, pH 2.0, containing 0.5M NaCI and 0.5-2% (v/v) Elugent™ detergent. Fractions containing rTbps were roughly located by monitoring the absorbance at 280 nm. As the ElugentM also absorbs at 280 nm, the presence of rTbps in selected fractions was confirmed by human transferrin-HRP (hTf-HRP) ligand blot and SDS-PAGE analysis. Fractions containing rTbps were pooled and applied to a HiPrep Desalting column (Sephadex G-25, Pharmacia) to partially remove glycine and free Elugen™. The protein concentration was then determined using the BCA kit (Pierce) using bovine serum albumin as the standard.

6. Transferrin-HRP Ligand Blot

Transferrin-HRP ligand blot was carried out to accurately locate the presence of rTbps in eluted fractions and confirmed that active protein was being recovered.

A series of eight two-fold dilutions was prepared using a 50 ul sample of each selected fraction and 5 ul of each spotted onto nitrocellulose membrane. The membrane was blocked with PBS containing 0.05% Tween 20 (PBST) and 1% (w/v) dried skimmed milk powder for 1 hour. After washing in PBST for 3×10 min the membrane was incubated in hTf-HRP conjugate (Jackson Immunoresearch Laboratories) diluted to 1 ug/ml in PBST for 2 h. After further washing, as above, the membrane was developed in 4-chloronaphthol substrate.

7. Results

7.1 Yields of Tbps

A range of detergents was investigated and compared to Elugent for their ability to solubilise and stabilise the rTbps. Of the nine examined octyl glucopyranoside and dodecyl maltoside were the most suitable alternatives for use.

The yields of rTbps from E. coli JM109 clones containing CAMR pMTL expression vectors were as follows:—

| | | | |
|---|---|---|---|
| | wet weight | | protein/litre |
| Vector | cells/litre | rTbp/g cells | culture |
| (pMTL) | culture (g) | (ug/g) | (mg) |

rTbpA

| | | | |
|---|---|---|---|
| 2000 | 19 | 306 | 5.8 |
| 2003 | 52* | 252 | ND |
| 2010 | 23 | 350 | 8.0 |
| 2010 | 22 | 537 | 11.8 |
| (no tet) | | | |

rTbpB

| | | | |
|---|---|---|---|
| 2000 | 33 | 880 | 29.0 |
| 2003 | 29 | 238 | 6.9 |
| 2010 | 42 | 690 | 28.9 |
| 2010 | 35 | 890 | 31.1 |
| (no tet) | | | |

* this cell pellet was very sloppy due to cell lysis and the wet weight value is therefore an over estimate.
ND - this value could not be calculated as actual wet weight is not known.

Samples of E. coli JM109 clones expressing, respectively, tbpA and tbpB were deposited at ECACC on Jan. 24, 2000 under accession numbers 00012404 and 00012405, respectively.

Yields were estimated from 40 g samples of cells taken from paste grown in 2× soytone medium in 8 litre fermenters. Tbps were extracted at 4° C. using procedure A and purified on a transferrin-Sepharose column. Protein concentrations were estimated using the BCA assay. Vectors pMTL2000 and pMTL2003 have the ampicillin resistance marker and vector pMTL2010 has the tetracycline resistance marker. Values in the last row for each Tbp with pMTL2010 are for no antibiotic present in the medium.

7.2 Characterisation of rTbps

7.2.1 SDS-PAGE Analysis of Purified rTbps

Purified rTbps were analysed by SDS-PAGE (10%) under different denaturing conditions (FIG. 1). Full length rTbpA had a MW of approximately 100 kDa and constituted >90% of total protein as determined by densitometry of the coomassie stained gel. When not heated before SDS-PAGE two major bands of slightly lower MW were apparent which may represent membrane inserted and unprocessed forms of rTbpA. Full length rTbpB had a MW of approximately 85 kDa and constituted ˜80% of total protein as determined by densitometry. There was no difference in the SDS-PAGE profile of the heated and unheated protein.

7.2.2 Western Blot of rTbps

After SDS-PAGE, purified rTbps were transferred electrophoretically to nitrocellulose membrane and probed with antibodies raised against native Tbps or hTf-HRP conjugate (FIG. 2). Like the native proteins purified from N. meningitidis, rTbpB binds hTf-HRP after SDS-PAGE and Western blot whereas rTbpA does not. A consistent pattern of low MW bands is seen for both rTbpA and rTbpB. These appear immediately after induction and when strains are grown at low temperatures and are most likely the result of in vivo proteolytic degradation of the full length rTbps. If they can be characterised and quantified they may not need to be removed from a vaccine preparation and may indeed contribute to protection afforded by the recombinant proteins.

7.3 Protection Provided by Recombinant Tbps Against Meningococcal Challenge in Mice.

Harlan-NIH mice were immunised on days 0, 21 and 28 with lOpg per dose of rTbpA, r rTbp or rTbpA+rTbpB in Freund's adjuvant. Mice were then given an intraperitoneal challenge of N. meningitidis strain K454 containing 10 mg of iron-saturated human transferrin on day 35. A further dose of human transferrin is given by intraperitoneal injection after 24 h. The numbers of surviving mice are then recorded for 4 days.

Neisseria meningiditis Strain K454 tbpA Gene DNA Sequence

| | |
|---|---|
| ATGCAACAGCAACATTTGTTCCGATTCAATATTTTATGCC | 40 |
| TGTCTTTAATGACTGCGCTGCCCGCTTATGCAGAAAATGT | 80 |
| GCAAGCCGGACAAGCACAGGAAAAACAGTTGGATACCATA | 120 |
| CAGGTAAAAGCCAAAAAACAGAAAACCCGCCGCGATAACG | 160 |
| AAGTAACCGGGCTGGGCAAGTTGGTCAAGTCTTCCGATAC | 200 |
| GCTAAGTAAAGAACAGGTTTTGAATATCCGAGACCTGACC | 240 |
| CGTTATGATCCGGGTATTGCCGTGGTCGAACAGGGTCGGG | 280 |
| GCGCAAGTTCCGGCTATTCAATACGCGGCATGGATAAAAA | 320 |
| CCGCGTTTCCTTAACGGTGGACGGCGTTTCGCAAATACAG | 360 |
| TCCTACACCGCGCAGGCGGCATTGGGCGGGACGAGGACGG | 400 |
| CGGGCAGCAGCGGCGCAATCAATGAAATCGAGTATGAAAA | 440 |
| CGTCAAAGCTGTCGAAATCAGCAAAGGCTCAAACTCGGTC | 480 |
| GAACAAGGCAGCGGCGCATTGGCGGGCTCGGTCGCATTTC | 520 |
| AAACCAAAACCGCCGACGATGTTATCGGGGAAGGCAGGCA | 560 |
| GTGGGGCATTCAGAGTAAAACCGCCTATTCCGGCAAAAAC | 600 |
| CGGGGGCTTACCCAATCCATCGCGCTGGCGGGGCGCATCG | 640 |
| GCGGTGCGGAGGCTTTGCTGATCCACACCGGGCGGCGCGC | 680 |
| GGGGGAAATCCGCGCCCACGAAGATGCAGGACGCGGCGTT | 720 |
| CAGAGCTTTAACAGGCTGGTGCCGGTTGAAGACAGCAGCA | 760 |
| ATTACGCCTATTTCATCGTTAAAGAAGAATGCAAAAACGG | 800 |
| GAGTTATGAAACGTGTAAAGCGAATCCCAAAAAAGATGTT | 840 |
| GTCGGCAAAGACGAACGTCAAACGGTTTCCACCCGAGACT | 880 |
| ACACGGGTCCCAACCGCTTCCTCGCCGATCCGCTTTCATA | 920 |
| CGAAAGCCGGTCGTGGCTGTTCCGCCCGGGTTTTCGTTTT | 960 |
| GAGAATAAGCGGCACTACATCGGCGGCATACTCGAACACA | 1000 |
| CGCAACAAACTTTCGACACGCGCGATATGACGGTTCCGGC | 1040 |
| ATTCCTGACCAAGGCGGTTTTTGATGCAAATAAAAAACAG | 1080 |
| GCGGGTTCTTTGCCCGGTAACGGCAAATACGCGGGCAACC | 1120 |
| ACAAATACGGCGGACTGTTTACCAACGGCGAAAACGGTGC | 1160 |
| GCTGGTGGGCGCGGAATACGGTACGGGCGTGTTTTACGAC | 1200 |
| GAGACGCACACCAAAAGCCGCTACGGTTTGGAATATGTCT | 1240 |
| ATACCAATGCCGATAAAGACACTTGGGCGGATTATGCCCG | 1280 |
| CCTCTCTTACGACCGGCAGGGCGTCGGTTTGGATAATCAT | 1320 |
| TTTCAGCAGACGCACTGTTCTGCCGACGGTTCGGACAAAT | 1360 |
| ATTGCCGCCCGAGTGCCGACAAGCCGTTTTCCTATTACAA | 1400 |
| ATCCGATCGCGTGATTTACGGGGAAAGCCACAGGCTCTTG | 1440 |
| CAGGCGGCATTCAAAAAATCCTTCGATACCGCCAAAATCC | 1480 |
| GCCACAACCTGAGCGTGAATCTCGGGTTTGACCGCTTTGG | 1520 |
| CTCTAATCTCCGCCATCAGGATTATTATTATCAACATGCC | 1560 |
| AACCGCGCCTATTCGTCGAACACGCCCCCTCAAAACAACG | 1600 |
| GCAAAAAAATCAGCCCCAACGGCAGTGAAACCAGCCCCTA | 1640 |
| TTGGGTCACCATAGGCAGGGGAAATGTCGTTACGGGGCAA | 1680 |
| ATCTGCCGCTTGGGCAACAATACTTATACGGACTGCACGC | 1720 |
| CGCGCAGCATCAACGGTAAAAGCTATTACGCGGCAGTTCG | 1760 |
| GGACAATGTCCGTTTGGGCAGGTGGGCGGATGTCGGCGCG | 1800 |
| GGCTTGCGCTACGACTACCGCAGCACGCATTCGGACGACG | 1840 |
| GGAGCGTTTCCACCGGCACGCACCGCACCTTGTCCTGGAA | 1880 |
| CGCCGGCATCGTCCTCAAACCTACCGACTGGCTGGATTTG | 1920 |
| ACTTACCGCACCTCAACCGGCTTCCGCCTGCCCTCGTTTG | 1960 |
| CGGAAATGTACGGCTGGCGGGCGGGTGTTCAAAGCAAGGC | 2000 |
| GGTCAAAATCGATCCGGAAAAATCGTTCAACAAAGAAGCC | 2040 |
| GGCATCGTGTTTAAAGGCGATTTCGGCAACTTGGAGGCAA | 2080 |
| GTTGGTTCAACAATGCCTACCGCGATTTGATTGTCCGGGG | 2120 |
| TTATGAAGCGCAAATTAAAGACGGCAAAGAAGAAGCCAAA | 2160 |
| GGCGACCCGGCTTACCTCAATGCCCAAAGCGCGCGGATTA | 2200 |
| CCGGCATCAATATTTTGGGCAAAATCGATTGGAACGGCGT | 2240 |
| ATGGGATAAATTGCCCGAAGGTTGGTATTCTACATTTGCC | 2280 |
| TATAATCGTGTCCGTGTCCGCGACATCAAAAAACGCGCAG | 2320 |
| ACCGCACCGATATTCAATCACATCTGTTTGATGCCATCCA | 2360 |
| ACCCTCGCGCTATGTCGTCGGCTTGGGCTATGACCAACCG | 2400 |
| GAAGGCAAATGGGGTGTGAACGGTATGCTGACTTATTCCA | 2440 |
| AAGCCAAGGAAATCACAGAGTTGTTGGGCAGCCGGGCTTT | 2480 |
| GCTCAACGGCAACAGCCGCAATACAAAAGCCACCGCGCGC | 2520 |
| CGTACCCGCCCTTGGTATATTGTGGACGTGTCCGGTTATT | 2560 |
| ACACGGTTAAAAAACACTTTACCCTCCGTGCGGGCGTGTA | 2600 |
| CAACCTCCTCAACTACCGCTATGTTACTTGGGAAAATGTG | 2640 |
| CGGCAAACTGCCGGCGGCGCAGTCAACCAACACAAAAATG | 2680 |
| TCGGCGTTTACAACCGATATGCCGCCCCCGGTCGCAACTA | 2720 |
| CACATTTAGCTTGGAAATGAAGTTTTAA | 2748 |

Translation of the Neisseria meningiditis Strain K454 tbpA Gene DNA Sequence

| | |
|---|---|
| MQQQHLFRFNILCLSLMTALPAYAENVQAGQAQEKQLDTI | 40 |
| QVKAKKQKTRRDNEVTGLGKLVKSSDTLSKEQVLNIRDLT | 80 |
| RYDPGIAVVEQGRGASSGYSIRGMDKNRVSLTVDGVSQIQ | 120 |
| SYTAQAALGGTRTAGSSGAINEIEYENVKAVETSKGSNSV | 160 |
| EQGSGALAGSVAFQTKTADDVIGEGRQWGIQSKTAYSGKN | 200 |
| RGLTQSIALAGRIGGAEALLIHTGRRAGEIRAHEDAGRGV | 240 |
| QSFNRLVPVEDSSNYAYFIVKEECKNGSYETCKANPKKDV | 280 |
| VGKDERQTVSTRDYTGPNRFLADPLSYESRSWLFRPGFRF | 320 |
| ENKRHYIGGILEHTQQTFDTRDMTVPAFLTKAVFDANKKQ | 360 |
| AGSLPGNGKYAGNHKYGGLFTNGENGALVGAEYGTGVFYD | 400 |
| ETHTKSRYGLEYVYTNADKDTWADYARLSYDRQGVGLDNH | 440 |
| FQQTHCSADGSDKYCRPSADKPFSYYKSDRVIYGESHRLL | 480 |
| QAAFKKSFDTAKIRHNLSVNLGFDRFGSNLRHQDYYYQHA | 520 |
| NRAYSSNTPPQNNGKKISPNGSETSPYWVTIGRGNVVTGQ | 560 |
| ICRLGNNTYTDCTPRSINGKSYYAAVRDNVRLGRWADVGA | 600 |
| GLRYDYRSTHSDDGSVSTGTHRTLSWNAGIVLKPTDWLDL | 640 |
| TYRTSTGFRLPSFAEMYGWRAGVQSKAVKIDPEKSFNKEA | 680 |
| GIVFKGDFGNLEASWFNNAYRDLIVRGYEAQIKDGKEEAK | 720 |
| GDPAYLNAQSARITGINILGKIDWNGVWDKLPEGWYSTFA | 760 |
| YNRVRVRDIKKRADRTDIQSHLFDAIQPSRYVVGLGYDQP | 800 |
| EGKWGVNGMLTYSKAKEITELLGSRALLNGNSRNTKATAR | 840 |
| RTRPWYIVDVSGYYTVKKHFTLRAGVYNLLNYRYVTWENV | 880 |
| RQTAGGAVNQHKNVGVYNRYAAPGRNYTFSLEMKF | 915 |

| Molecular Weight 102091.85 Daltons | |
|---|---|
| 915 Amino Acids | |
| 124 Strongly Basic(+) Amino Acids (K, R) | |
| 93 Strongly Acidic(−) Amino Acids (D, E) | |
| 274 Hydrophobic Amino Acids (A, I, L, F, W, V) | |
| 282 Polar Amino Acids (N, C, Q, S, T, Y) | |
| 9.472 Isoelectric Point | |
| 33.723 Charge at pH 7.0 | |
| Total number of bases translated is 2748 | |
| % A = 2635 | [724] |
| % G = 26.67 | [733] |
| % T = 20.23 | [556] |
| % C = 26.75 | [735] |
| % Ambiguous = 0.00 | [0] |
| % A + T = 46.58 | [1280] |
| % C + G = 53.42 | [1468] |

Neisseria meningiditis Strain K454 tbpB Gene DNA Sequence

| | |
|---|---|
| ATGAACAATCCATTGGTGAATCAGGCTGCTATGGTGCTGC | 40 |
| CTGTGTTTTTGTTGAGTGCTTGTTTGGGCGGAGGCGGCAG | 80 |
| TTTCGATCTTGATTCTGTCGATACCGAAGCCCCGCGTCCC | 120 |
| GCGCCAAAATATCAAGATGTTTTTTCCGAAAAACCGCAAG | 160 |
| CCCAAAAAGACCAAGGCGGATACGGTTTTGCAATGAGGTT | 200 |
| GAAACGGAGGAATTGGTATCCGCAGGCAAAAGAAGACGAG | 240 |
| GTTAAACTGGACGAGAGTGATTGGGAGGCGACAGGATTGC | 280 |
| CGGACGAACCTAAGGAACTCCCTAAACGGCAAAAATCGGT | 320 |
| TATCGAAAAAGTAGAAACAGACAGCGACAACAATATTTAT | 360 |
| TCTTCCCCCTATCTCAAACCATCAAACCATCAAAACGGCA | 400 |
| ACACTGGCAACGGTATAAACCAACCTAAAAATCAGGCAAA | 440 |
| AGATTACGAAAATTTTAAATATGTTTATTCCGGCTGGTTT | 480 |
| TACAAACACGCCAAACGAGAGTTTAACTTAAAGGTGGAAC | 520 |
| CTAAAAGTGCAAAAAACGGCGACGACGGTTATATCTTCTA | 560 |
| TCACGGTAAAGAACCTTCCCGACAACTTCCCGCTTCTGGA | 600 |
| AAAATTACCTATAAAGGTGTGTGGCATTTTGCGACCGATA | 640 |
| CAAAAAAGGGTCAAAAATTTCGTGAAATTATCCAACCTTC | 680 |
| AAAAAGTCAAGGCGACAGGTATAGCGGATTTTCGGGCGAT | 720 |
| GACGGCGAAGAATATTCCAACAAAAACAAATCCACGCTGA | 760 |
| CAGATGGTCAAGAGGGTTATGGTTTTACCTCAAATTTAGA | 800 |
| AGTGGATTTCCATAATAAAAAATTGACGGGCAAACTGATA | 840 |
| CGCAACAATGCGAATACCGATAACAACCAAGCCACCACCA | 880 |
| CGCAATACTACAGCCTTGAGGCTCAAGTAACAGGCAACCG | 920 |
| CTTCAACGGCAAGGCAACGGCAACCGACAAACCCCAACAA | 960 |
| AACAGCGAAACCAAGGAACATCCCTTTGTTTCCGATTCGT | 1000 |
| CTTCTTTGAGCGGCGGCTTTTTCGGCCCGCAGGGTGAGGA | 1040 |
| ATTGGGTTTCCGCTTTTTGAGCGACGATCAAAAAGTTGCC | 1080 |
| GTTGTCGGCAGCGCGAAAACCAAAGACAAACCCGCAAATG | 1120 |
| GCAATACTGCGGCGGCTTCAGGCGGCACAGATGCGGCAGC | 1160 |
| ATCAAACGGTGCGGCAGGCACGTCGTCTGAAAACGGTAAG | 1200 |
| CTGACCACGGTTTTGGATGCGGTCGAGCTGAAATTGGGCG | 1240 |
| ATAAGAAAGTCCAAAAGCTCGACAACTTCAGCAACGCCGC | 1280 |
| CCAACTGGTTGTCGACGGCATTATGATTCCGCTCTTGCCC | 1320 |
| GAGGCTTCCGAAAGTGGGAACAATCAAGCCAATCAAGGTA | 1360 |
| CAAATGGCGGAACAGCCTTTACCCGCAAATTTGACCACAC | 1400 |
| GCCGGAAAGTGATAAAAAAGACGCCCAAGCAGGTACGCAG | 1440 |
| ACGAATGGGGCGCAAACCGCTTCAAATACGGCAGGTGATA | 1480 |
| CCAATGGCAAAACAAAAACCTATGAAGTCGAAGTCTGCTG | 1520 |
| TTCCAACCTCAATTATCTGAAATACGGAATGTTGACGCGC | 1560 |
| AAAAACAGCAAGTCCGCGATGCAGGCAGGAGAAAGCAGTA | 1600 |
| GTCAAGCTGATGCTAAAACGGAACAAGTTGAACAAAGTAT | 1640 |
| GTTCCTCCAAGGCGAGCGCACCGATGAAAAAGAGATTCCA | 1680 |
| AGCGAGCAAAACATCGTTTATCGGGGGTCTTGGTACGGAT | 1720 |
| ATATTGCCAACGACAAAAGCACAAGCTGGAGCGGCAATCC | 1760 |
| TTCCAATGCAACGAGTGGCAACAGGGCGGAATTTACTGTG | 1800 |
| AATTTTGCCGATAAAAAAATTACTGGTACGTTAACCGCTG | 1840 |
| ACAACAGGCAGGAGGCAACCTTTACCATTGATGGTAATAT | 1880 |
| TAAGGACAACGGCTTTGAAGGTACGGCGAAAACTGCTGAG | 1920 |
| TCAGGTTTTGATCTCGATCAAAGCAATACCACCCGCACGC | 1960 |
| CTAAGGCATATATCACAGATGCCAAGGTGCAGGGCGGTTT | 2000 |
| TTACGGGCCCAAAGCCGAAGACTTGGGCGGATGGTTTGCC | 2040 |
| TATCCGGGCGATAAACAAACGAAAAATGCAACAAATGCAT | 2080 |
| CCGGCAATAGCAGTGCAACTGTCGTATTCGGTGCGAAACG | 2120 |
| CCAACAGCCTGTGCAATAA | 2139 |

Translation of the Neisseria meningiditis Strain K454 tbpB Gene DNA Sequence

| | |
|---|---|
| MNNPLVNQAAMVLPVFLLSACLGGGGSFDLDSVDTEAPRP | 40 |
| APKYQDVFSEKPQAQKDQGGYGFAMRLKRRNWYPQAKEDE | 80 |
| VKLDESDWEATGLPDEPKELPKRQKSVIEKVETDSDNNIY | 120 |
| SSPYLKPSNHQNGNTGNGINQPKNQAKDYENFKYVYSGWF | 160 |
| YKHAKREFNLKVEPKSAKNGDDGYIFYHGKEPSRQLPASG | 200 |
| KITYKGVWHFATDTKKGQKFREIIQPSKSQGDRYSGFSGD | 240 |
| DGEEYSNKNKSTLTDGQEGYGFTSNLEVDFHNKKLTGKLI | 280 |
| RNNANTDNNQATTTQYYSLEAQVTGNRFNGKATATDKPQQ | 320 |
| NSETKEHPFVSDSSSLSGGFFGPQGEELGFRFLSDDQKVA | 360 |
| VVGSAKTKDKPANGNTAAASGGTDAAASNGAAGTSSENGK | 400 |
| LTTVLDAVELKLGDKKVQKLDNFSNAAQLVVDGIMIPLLP | 440 |
| EASESGNNQANQGTNGGTAFTRKFDHTPESDKKDAQAGTQ | 480 |
| TNGAQTASNTAGDTNGKTKTYEVEVCCSNLNYLKYGMLTR | 520 |
| KNSKSAMQAGESSSQADAKTEQVEQSMFLQGERTDEKEIP | 560 |
| SEQNIVYRGSWYGYIANDKSTSWSGNASNATSGNRAEFTV | 600 |
| NFADKKITGTLTADNRQEATFTIDGNIKDNGFEGTAKTAE | 640 |
| SGFDLDQSNTTRTPKAYITDAKVQGGFYGPKAEELGGWFA | 680 |
| YPGDKQTKNATNASGNSSATVVFGAKRQQPVQ | 712 |

| Molecular Weight 77386.47 Daltons | |
|---|---|
| 712 Amino Acids | |
| 84 Strongly Basic(+) Amino Acids (K, R) | |
| 90 Strongly Acidic(−) Amino Acids (D, E) | |
| 184 Hydrophobic Amino Acids (A, I, L, F, W, V) | |
| 241 Polar Amino Acids (N, C, Q, S, T, Y) | |
| 6.000 Isoelectric Point | |
| −4.964 Charge at PH 7.0 | |
| Total number of bases translated is 2139 | |
| % A = 32.54 | [696] |
| % G = 24.26 | [519] |
| % T = 20.20 | [432] |
| % C = 23.00 | [492] |
| % Ambiguous = 0.00 | [0] |
| % A + T = 52.73 | [1128] |
| % C + G = 47.27 | [1011] |

### EXAMPLE 2

Recombinant Human Transferrin Expression in E. coli

As an alternative to using human blood derived transferrin for the purification of rTbps, we have expressed a recombinant form of human transferrin in E. coli. We have expressed individual lobes of the transferrin protein, along with full length protein.

Human transferrin was cloned by PCR amplification of an existing gene clone (cDNA sequence Funmei Yang et al., (1984) PNAS 81: 2752-2756). Before use, the internal NdeI sites present in the hTf gene were removed by mutagenic PCR, as follows:

1. PCR amplification of the transferrin with the oligomers below removed the first NdeI site at amino acids 25-26, without changing the amino acid sequence. An NruI site is included in the 5′ primer, enabling the product to be cloned into a previously engineered version of hTf containing an NruI site just upstream of the NdeI site (also engineered without changing the amino acid sequence).

Primers for Removing 5′ (Upstream) NdeI Site

5′TTT CGC GAG GAG ATG AAA AGO GTC ATT OCA TCC 3′
(5′primer)
5′GTT CTA GAG TGG GAG CCC TAC CTC TGA G 3′
(3′primer)

2. Removal of the second NdeI site was a two step process: firstly, a version of hTf was generated containing an appropriately placed PvuI site in it (amino acids 642-645). The PvuI site was introduced by PCR amplification of hTf lacking the upstream NdeI site (generated as detailed above) with the following oligomers

Primers for Introducing PvuI Site into hTf

5′CAT ATG GTC CCT GAT AAA ACT GTG AG 3′
(5′primer)
5′CGA TCG TGA AGT TTG GCC AAA CAT ACT G 3′
(3′primer)

Then the 3′ end of hTf was amplified using the following oligomers:

Primers for Removing 3′ (Downstream) NdeI Site

5′CGA TCG AAA CAG GTA TGA AAA ATA CTT AG 3′
(5′primer)
5′GTT CTA GAG TGG CAG CCC TAC CTC TGA G 3′
(3′primer)

3. The PvuI sites were used to join the two products together, forming a full length recombinant hTf gene with a single NdeI site at the level of the start ATG codon.

The N terminal clone was prepared by PCR, using the oligomers below, generating an N terminus clone without the native leader sequence, encompassing amino acids 1-337 of the mature transferrin sequence.

N Terminus Clone Primers

5′CAT ATG GTC CCT GAT AAA ACT GTG AG 3′
(5′primer)
5′TCT AGA TTA ATC TGT TGG GGC TTC TGG GCA TG 3′
(3′primer)

The C terminal lobe was amplified using the oligomers below, which again enabled cloning into the NdeI site of pET and pMTL vectors, and encompassed amino acids 338-679 of the mature transferrin sequence.

C Terminus Clone Primers

5′CAT ATG GAA TGC AAG CCT GTG AAG TGG 3′
(5′primer)
5′GTT CTA GAG TGG CAG CCC TAC CTC TGA G 3′
(3′primer)

Full-length and hTf lobes were cloned into pET22b and pET26b, initially, on an NdeI-XbaI fragment.

Expression Studies

Expression studies were carried out by growing E. coli BL21 DE3 carrying the hTf pET22b and pET26b clones, to OD₆₀₀ 0.7-1.0. Expression was induced with 1 mM IPTG and hTf production monitored over the course of two hours by dot blot and Western blotting, using a goat anti-human transferrin polyclonal antibody (Sigma). The size of full length and C terminus recombinant matched that expected for unglycosylated human transferrin and its individual lobes. Microscope examination revealed that expression of hTf resulted in the production of inclusion bodies. This precipitated material requires solubilisation and refolding in order to generate functional material.

### EXAMPLE 3

Recombinant Transferrin Refolding and Preparation of Affinity Column

The protocol for solubilisation and refolding is described in (Hoefkins P., et al. (1996) Int. J. Biochem. Cell. Biol. 28, 975-982. Briefly, the protocol is:—

1. Isolate inclusion body material by standard cell lysis and centrifugation.

2. Dissolve pelleted protein in 8M urea, 1 mM DTT, 40 mM Tris/HCl, 10% glycerol (v/v) pH 7.6.

3. Dilute dissolved protein in renaturation buffer (0.1 mM Na-EDTA, 0.1 mM Tris/HCl, 1.0 mM reduced glutathione (GSH), pH 8.2) to a concentration of 20 μg/ml.

4. Incubate at 6° C. for 15 min.

5. Add oxidised glutathione (GSSG) to a final concentration of 0.5 mM.

6. Incubate for further 22 hr at 6° C.

7. Concentrate and dialyse against 10 mM NaHCO₃.

8. Saturate with iron and assess purity (where necessary, clean up using size exclusion chromatography or other chromatographic technique).

9. Conjugate with Sepharose 4B (Amersham Pharmacia) to generate affinity matrix.

The resultant transferrin affinity column is used to purify recombinant Tbps from Example 1.

### EXAMPLE 4

Protective Effect of Recombinant Tbps

Groups of 20 mice were inoculated with rTbpA, rTbpB, both rTbpA and rTbpB, or a control of no vaccine. Their survival was monitored following challenge by 5×10⁶ and 5×10⁷ N. meningitidis strain K454 and the results illustrated in FIG. 3.

rTbpA and rTbpB conferred protection against challenge, confirming antigenicity of native Tbp had been retained in the recombinant proteins. In more detail, both rTbpA and rTbpB provide strong protection against meningococcal challenge, with greater protection provided by TbpA at the higher challenge dose: Protection with TbpA has not been previously reported. The combination of TbpA+TbpB is also protective and may provide the most effective vaccine against a range of challenge strains.

### EXAMPLE 5

Surface Expression of Recombinant Tbps

E. coli expressing recombinant Tbps are probed with fluorescently labelled human transferrin, with the results being shown in FIG. 4.

It is seen that the parent E. coli strain and the uninduced strains possessing the Tbp gene show little fluorescence. The induced E. coli peak is shifted to the right on the X-axis, indicating increased fluorescence caused by binding of the labelled human transferin to the bacteria, indicating location of the recombinant Tbps on the bacterial surface.

### EXAMPLE 6

Cross-Reactivity of Antisera Raised Against rTbpA and rTbpB

Whole-cell ELISA studies are carried out using a range of patient isolates collected in Norway to assess the cross-reactivity of antisera raised against rTbpA and rTbpB with Tbps expressed by these isolates, with the results being shown in table 2.

TABLE 2
Meningococcal whole-cell ELISA titers of sera
from mice immunized with rTpbA and/or rTbpB

| | | | Whole-cell ELISA | |
|---|---|---|---|---|
| | | | titers^{c} in | |
| Meningococcal strain details | | | immunization group: | |
| Serotype or group | Isolate Source^{a} | TbpB type^{b} | TpbA | TbpB |
| B:15P1.7, 16 | 6 | H | 19,676 | 3,050 |
| B:15:P1.7, 16 | 8 | H | 20,650 | 3,667 |
| C:15P1.7, 16 | 9 | H | 4,987 | 614 |
| C:15P1.7, 16 | 12 | H | 9,726 | 1,267 |
| C:15:P1.7, 16 | 13 | H | 7,514 | 1,389 |
| C:15:P1.7, 16 | 14 | H | 2,301 | 709 |
| B:15:P1.2 | 20 | H | 3,893 | 1,283 |
| B:15:P1.12V | 22 | H | 16,364 | 3,488 |
| B:15:P1.12V | 23 | H | 11,903 | 1,858 |
| C:2a:P1.2 | 29 | H | 7,785 | 3,269 |
| B:NT:P1.12 | 32 | H | 17,385 | 10,226 |
| B:NT:P1.16 | 33 | H | 9,614 | 339 |
| B:4:P1.12 | 37 | H | 18,653 | 7,461 |
| B:19:P1.15 | 39 | H | 18,108 | 1,609 |
| C:2a:P1.2 | 26 | L | 2,539 | 216 |
| C:2a:P1.2 | 27 | L | 12,116 | 1,314 |
| C:2a:P1.2 | 28 | L | 172,779 | 1,130 |

^{a}Patient numbers were assigned in a previous study.
^{b}The molecular masses of TbpB are grouped as follows: H (high, >80 kDa) or L (low, <70 kDa).
^{c}Geometric mean of the reciprocal titer from five separate animals. Where no antibody was detected (e.g., preimmune sera [data not shown]), an arbitrary titer of 50 was assigned. The mean values are 10,408 for the TbpA group and 1,573 for the TbpB group.

It is seen that titers against isolates of a variety of different sera groups, sera types and serosubtypes were consistently higher for antisera raised against rTbpA than for antisera raised against rTbpB.

Three isolates expressing low-molecular mass TbpB and the rTbpB antiserum showed reaction with these cells. Pre-immune sera from these mice showed no reaction with the meningococcal isolates.

The invention thus provides recombinant expression of iron uptakes proteins and compositions, vaccines and uses based thereon.

1. A non-neisserial cell expressing a neisserial iron uptake protein, wherein the iron uptake protein can be extracted from the cell under mild conditions and retains substantially the antigenicity of native iron uptake protein.

2. A non-neisserial cell expressing a neisserial iron uptake protein wherein the iron uptake protein is located in an outer surface membrane of the cell.

3. A cell overexpressing an iron uptake protein, wherein the iron uptake protein is located in an outer surface membrane of the cell.

4. A non-neisserial cell according to any of claims 1 to 3 expressing a neisserial transferrin binding protein (Tbp) wherein:—
said Tbp can be extracted from the cell under mild conditions; and
said extracted Tbp retains substantially the antigenicity of native TbpA.

5. A bacterial cell according to any of claims 1 to 4, expressing TbpA.

6. A bacterial cell according to any of claims 1 to 5, expressing TbpB.

7. A method of producing an iron uptake protein by expressing a recombinant iron uptake protein gene in a non-neisserial cell host such that the protein is expressed and translocated to a surface membrane of the host.

8. A method according to claim 7 for producing a neisserial iron uptake protein selected from the group consisting of transferrin binding proteins (Tbps), lactoferrin binding proteins (Lbps), haemoglobin binding protein, enterobactin binding protein, vibriobactin binding protein, ferric siderophore binding protein, heme binding protein, hemin binding protein, chrysobactin binding protein, hydroxymate binding protein and pseudobactin binding protein.

9. A method of producing a neisserial transferrin binding protein (Tbp) comprising:
a. expressing a recombinant neisserial Tbp gene in a non-neisserial host such that Tbp protein is expressed and translocated to the cell membrane;
b. under mild conditions, extracting the Tbp protein; and, optionally
c. purifying said Tbp protein.

10. A method according to claim 9 wherein the cell expresses TbpA.

11. A method according to claim 9 or 10 wherein the cell expresses TbpB.

12. A method according to any of claims 7 to 11 comprising extracting the Tbp by solubilising membrane bound Tbp in a non-ionic detergent solution.

13. A method according to claim 12 wherein said non-ionic detergent is selected from the group consisting of an alkyl glucoside; n-octyl-β-D-glucopyranoside; TRITON® X100; ELUGENT®; dodecyl-maltoside; and n-octyl-β-maltoside.

14. A method according to claim 13 comprising a low energy homogenisation step.

15. A method according to any of claims 7 to 14, comprising breaking cells and cell membranes with a bead-beating apparatus.

16. A method according to any of claims 7 to 15 wherein said native Tbp protein is purified by affinity chromatography.

17. A method according to claim 16 wherein said affinity chromatography comprises using a transferrin-bound affinity matrix.

18. A method according to claim 17 wherein said transferrin is human transferrin.

19. A method according to any of claims 7 to 18 comprising culturing a cell according to any of claims 1 to 6.

20. A method of preparing a vaccine, comprising obtaining an iron uptake protein according to any of claims 7 to 19 and combining said protein with a pharmaceutically acceptable carrier.

21. Use of a cell according to any of claims 1 to 6 in manufacture of Tbp.

22. Use of a cell according to any of claims 1 to 6 in manufacture of a vaccine for protection against neisserial disease and/or meningococcal disease and/or gonococcal disease.

23. An expression construct comprising a nucleotide sequence encoding an iron uptake protein and a leader sequence directing the expressed protein to a surface membrane of the host.

24. An expression construct according to claim 23, wherein the leader sequence comprises all or part of a neisserial leader sequence.

25. An expression construct according to claim 23 or 24, wherein the leader sequence is the native leader sequence for the iron uptake protein.

26. An expression construct according to claim 23, for expression of the protein in a host cell, wherein the leader sequence is all or part of a leader sequence native to the host.

27. An affinity matrix for purification of Tbps comprising human transferrin or fragments thereof that bind Tbps.

28. An affinity matrix according to claim 27 comprising human recombinant transferrin or fragments thereof.

29. A method of purifying a Tbp, comprising eluting a Tbp-containing preparation through an affinity matrix comprising immobilized transferrin.

30. A method according to claim 29 using an affinity matrix according to claim 27 or 28.

31. A method according to claims 29 or 30 wherein the Tbp—containing preparation is obtained according to any of claims 7 to 19.

32. A commensal neisseria expressing an iron uptake protein from a pathogenic neisseria.

33. A commensal neisseria according to claim 32 expressing an iron uptake protein from a pathogenic Neisseria in an outer surface membrane of the commensal neisseria.

34. A composition containing a Tbp, wherein at least 90 percent by weight of said Tbp is active Tbp.

35. A composition according to claim 34, free of Tbp that is not capable of binding transferrin.

36. A culture of cells which express a neisserial iron uptake protein, wherein the yield of said protein is at least 4 mg per litre of culture.

37. A cell culture according to claim 36 wherein the protein is TbpA.

38. A cell culture according to claim 36 wherein the protein is TbpB.

39. A composition comprising an outer membrane vesicle preparation (OMV), wherein the OMV comprises an iron uptake protein and is prepared from a cell according to any of claims 1 to 6.

40. A method of making a composition for vaccination, comprising obtaining an OMV from a cell according to any of claims 1 to 6.

41. A method of producing a transferrin binding protein (Tbp) from a pathogenic Neisseria, comprising:—
d. expressing a gene encoding the Tbp in a commensal Neisserial host such that Tbp protein is translocated to an outer surface membrane of the commensal host;
e. extracting the Tbp under mild conditions; and
f. optionally, purifying said Tbp protein.

42. A method according to claim 41, comprising extracting an outer membrane vesicle preparation.

43. A method according to claim 41 or 42, comprising expressing a N. meningitidis gene in N. lactamica.

44. A method according to any of claims 41 to 43 wherein the Tbp is TbpA.

45. A method according to any of claims 41 to 43 wherein the Tbp is TbpB.

46. A method according to any of claims 41 to 45 comprising extracting the Tbp by solubilising membrane bound Tbp in a non-ionic detergent. solution.


SEQUENCE LISTING (18 sequences)

SEQ ID NO: 1 - DNA, 26 bp, Artificial Sequence
  primer
ttagggaaaccatatgcaacagcaac

SEQ ID NO: 2 - DNA, 31 bp, Artificial Sequence
  primer
gacggatccgcgtttggacgtttaaaacttc

SEQ ID NO: 3 - DNA, 25 bp, Artificial Sequence
  primer
gaattggatttcatatgaacaatcc

SEQ ID NO: 4 - DNA, 28 bp, Artificial Sequence
  primer
gacgaattccggcagccgtgcttatcgc

SEQ ID NO: 5 - DNA, 2748 bp, Neisseria meningitidis
atgcaacagcaacatttgttccgattcaatattttatgcctgtctttaatgactgcgctgcccgcttatgcagaaaatgtgcaagccggacaagcacaggaaaaacagttggataccatacaggtaaaagccaaaaaacagaaaacccgccgcgataacgaagtaaccgggctgggcaagttggtcaagtcttccgatacgctaagtaaagaacaggttttgaatatccgagacctgacccgttatgatccgggtattgccgtggtcgaacagggtcggggcgcaagttccggctattcaatacgcggcatggataaaaaccgcgtttccttaacggtggacggcgtttcgcaaatacagtcctacaccgcgcaggcggcattgggcgggacgaggacggcgggcagcagcggcgcaatcaatgaaatcgagtatgaaaacgtcaaagctgtcgaaatcagcaaaggctcaaactcggtcgaacaaggcagcggcgcattggcgggctcggtcgcatttcaaaccaaaaccgccgacgatgttatcggggaaggcaggcagtggggcattcagagtaaaaccgcctattccggcaaaaaccgggggcttacccaatccatcgcgctggcggggcgcatcggcggtgcggaggctttgctgatccacaccgggcggcgcgcgggggaaatccgcgcccacgaagatgcaggacgcggcgttcagagctttaacaggctggtgccggttgaagacagcagcaattacgcctatttcatcgttaaagaagaatgcaaaaacgggagttatgaaacgtgtaaagcgaatccgaaaaaagatgttgtcggcaaagacgaacgtcaaacggtttccacccgagactacacgggtcccaaccgcttcctcgccgatccgctttcatacgaaagccggtcgtggctgttccgcccgggttttcgttttgagaataagcggcactacatcggcggcatactcgaacacacgcaacaaactttcgacacgcgcgatatgacggttccggcattcctgaccaaggcggtttttgatgcaaataaaaaacaggcgggttctttgcccggtaacggcaaatacgcgggcaaccacaaatacggcggactgtttaccaacggcgaaaacggtgcgctggtgggcgcggaatacggtacgggcgtgttttacgacgagacgcacaccaaaagccgctacggtttggaatatgtctataccaatgccgataaagacacttgggcggattatgcccgcctctcttacgaccggcagggcgtcggtttggataatcattttcagcagacgcactgttctgccgacggttcggacaaatattgccgcccgagtgccgacaagccgttttcctattacaaatccgatcgcgtgatttacggggaaagccacaggctcttgcaggcggcattcaaaaaatccttcgataccgccaaaatccgccacaacctgagcgtgaatctcgggtttgaccgctttggctctaatctccgccatcaggattattattatcaacatgccaaccgcgcctattcgtcgaacacgccccctcaaaacaacggcaaaaaaatcagccccaacggcagtgaaaccagcccctattgggtcaccataggcaggggaaatgtcgttacggggcaaatctgccgcttgggcaacaatacttatacggactgcacgccgcgcagcatcaacggtaaaagctattacgcggcagttcgggacaatgtccgtttgggcaggtgggcggatgtcggcgcgggcttgcgctacgactaccgcagcacgcattcggacgacggcagcgtttccaccggcacgcaccgcaccttgtcctggaacgccggcatcgtcctcaaacctaccgactggctggatttgacttaccgcacctcaaccggcttccgcctgccctcgtttgcggaaatgtacggctggcgggcgggtgttcaaagcaaggcggtcaaaatcgatccggaaaaatcgttcaacaaagaagccggcatcgtgtttaaaggcgatttcggcaacttggaggcaagttggttcaacaatgcctaccgcgatttgattgtccggggttatgaagcgcaaattaaagacggcaaagaagaagccaaaggcgacccggcttacctcaatgcccaaagcgcgcggattaccggcatcaatattttgggcaaaatcgattggaacggcgtatgggataaattgcccgaaggttggtattctacatttgcctataatcgtgtccgtgtccgcgacatcaaaaaacgcgcagaccgcaccgatattcaatcacatctgtttgatgccatccaaccctcgcgctatgtcgtcggcttgggctatgaccaaccggaaggcaaatggggtgtgaacggtatgctgacttattccaaagccaaggaaatcacagagttgttgggcagccgggctttgctcaacggcaacagccgcaatacaaaagccaccgcgcgccgtacccgcccttggtatattgtggacgtgtccggttattacacggttaaaaaacactttaccctccgtgcgggcgtgtacaacctcctcaactaccgctatgttacttgggaaaatgtgcggcaaactgccggcggcgcagtcaaccaacacaaaaatgtcggcgtttacaaccgatatgccgcccccggtcgcaactacacatttagcttggaaatgaagttttaa

SEQ ID NO: 6 - protein, 915 aa, Neisseria meningitidis
MQQQHLFRFNILCLSLMTALPAYAENVQAGQAQEKQLDTIQVKAKKQKTRRDNEVTGLGKLVKSSDTLSKEQVLNIRDLTRYDPGIAVVEQGRGASSGYSIRGMDKNRVSLTVDGVSQIQSYTAQAALGGTRTAGSSGAINEIEYENVKAVEISKGSNSVEQGSGALAGSVAFQTKTADDVIGEGRQWGIQSKTAYSGKNRGLTQSIALAGRIGGAEALLIHTGRRAGEIRAHEDAGRGVQSFNRLVPVEDSSNYAYFIVKEECKNGSYETCKANPKKDVVGKDERQTVSTRDYTGPNRFLADPLSYESRSWLFRPGFRFENKRHYIGGILEHTQQTFDTRDMTVPAFLTKAVFDANKKQAGSLPGNGKYAGNHKYGGLFTNGENGALVGAEYGTGVFYDETHTKSRYGLEYVYTNADKDTWADYARLSYDRQGVGLDNHFQQTHCSADGSDKYCRPSADKPFSYYKSDRVIYGESHRLLQAAFKKSFDTAKIRHNLSVNLGFDRFGSNLRHQDYYYQHANRAYSSNTPPQNNGKKISPNGSETSPYWVTIGRGNVVTGQICRLGNNTYTDCTPRSINGKSYYAAVRDNVRLGRWADVGAGLRYDYRSTHSDDGSVSTGTHRTLSWNAGIVLKPTDWLDLTYRTSTGFRLPSFAEMYGWRAGVQSKAVKIDPEKSFNKEAGIVFKGDFGNLEASWFNNAYRDLIVRGYEAQIKDGKEEAKGDPAYLNAQSARITGINILGKIDWNGVWDKLPEGWYSTFAYNRVRVRDIKKRADRTDIQSHLFDAIQPSRYVVGLGYDQPEGKWGVNGMLTYSKAKEITELLGSRALLNGNSRNTKATARRTRPWYIVDVSGYYTVKKHFTLRAGVYNLLNYRYVTWENVRQTAGGAVNQHKNVGVYNRYAAPGRNYTFSLEMKF

SEQ ID NO: 7 - DNA, 2139 bp, Neisseria meningitidis
atgaacaatccattggtgaatcaggctgctatggtgctgcctgtgtttttgttgagtgcttgtttgggcggaggcggcagtttcgatcttgattctgtcgataccgaagccccgcgtcccgcgccaaaatatcaagatgttttttccgaaaaaccgcaagcccaaaaagaccaaggcggatacggttttgcaatgaggttgaaacggaggaattggtatccgcaggcaaaagaagacgaggttaaactggacgagagtgattgggaggcgacaggattgccggacgaacctaaggaactccctaaacggcaaaaatcggttatcgaaaaagtagaaacagacagcgacaacaatatttattcttccccctatctcaaaccatcaaaccatcaaaacggcaacactggcaacggtataaaccaacctaaaaatcaggcaaaagattacgaaaattttaaatatgtttattccggctggttttacaaacacgccaaacgagagtttaacttaaaggtggaacctaaaagtgcaaaaaacggcgacgacggttatatcttctatcacggtaaagaaccttcccgacaacttcccgcttctggaaaaattacctataaaggtgtgtggcattttgcgaccgatacaaaaaagggtcaaaaatttcgtgaaattatccaaccttcaaaaagtcaaggcgacaggtatagcggattttcgggcgatgacggcgaagaatattccaacaaaaacaaatccacgctgacagatggtcaagagggttatggttttacctcaaatttagaagtggatttccataataaaaaattgacgggcaaactgatacgcaacaatgcgaataccgataacaaccaagccaccaccacgcaatactacagccttgaggctcaagtaacaggcaaccgcttcaacggcaaggcaacggcaaccgacaaaccccaacaaaacagcgaaaccaaggaacatccctttgtttccgattcgtcttctttgagcggcggctttttcggcccgcagggtgaggaattgggtttccgctttttgagcgacgatcaaaaagttgccgttgtcggcagcgcgaaaaccaaagacaaacccgcaaatggcaatactgcggcggcttcaggcggcacagatgcggcagcatcaaacggtgcggcaggcacgtcgtctgaaaacggtaagctgaccacggttttggatgcggtcgagctgaaattgggcgataagaaagtccaaaagctcgacaacttcagcaacgccgcccaactggttgtcgacggcattatgattccgctcttgcccgaggcttccgaaagtgggaacaatcaagccaatcaaggtacaaatggcggaacagcctttacccgcaaatttgaccacacgccggaaagtgataaaaaagacgcccaagcaggtacgcagacgaatggggcgcaaaccgcttcaaatacggcaggtgataccaatggcaaaacaaaaacctatgaagtcgaagtctgctgttccaacctcaattatctgaaatacggaatgttgacgcgcaaaaacagcaagtccgcgatgcaggcaggagaaagcagtagtcaagctgatgctaaaacggaacaagttgaacaaagtatgttcctccaaggcgagcgcaccgatgaaaaagagattccaagcgagcaaaacatcgtttatcgggggtcttggtacggatatattgccaacgacaaaagcacaagctggagcggcaatgcttccaatgcaacgagtggcaacagggcggaatttactgtgaattttgccgataaaaaaattactggtacgttaaccgctgacaacaggcaggaggcaacctttaccattgatggtaatattaaggacaacggctttgaaggtacggcgaaaactgctgagtcaggttttgatctcgatcaaagcaataccacccgcacgcctaaggcatatatcacagatgccaaggtgcagggcggtttttacgggcccaaagccgaagagttgggcggatggtttgcctatccgggcgataaacaaacgaaaaatgcaacaaatgcatccggcaatagcagtgcaactgtcgtattcggtgcgaaacgccaacagcctgtgcaataa

SEQ ID NO: 8 - protein, 712 aa, Neisseria meningitidis
MNNPLVNQAAMVLPVFLLSACLGGGGSFDLDSVDTEAPRPAPKYQDVFSEKPQAQKDQGGYGFAMRLKRRNWYPQAKEDEVKLDESDWEATGLPDEPKELPKRQKSVIEKVETDSDNNIYSSPYLKPSNHQNGNTGNGINQPKNQAKDYENFKYVYSGWFYKHAKREFNLKVEPKSAKNGDDGYIFYHGKEPSRQLPASGKITYKGVWHFATDTKKGQKFREIIQPSKSQGDRYSGFSGDDGEEYSNKNKSTLTDGQEGYGFTSNLEVDFHNKKLTGKLIRNNANTDNNQATTTQYYSLEAQVTGNRFNGKATATDKPQQNSETKEHPFVSDSSSLSGGFFGPQGEELGFRFLSDDQKVAVVGSAKTKDKPANGNTAAASGGTDAAASNGAAGTSSENGKLTTVLDAVELKLGDKKVQKLDNFSNAAQLVVDGIMIPLLPEASESGNNQANQGTNGGTAFTRKFDHTPESDKKDAQAGTQTNGAQTASNTAGDTNGKTKTYEVEVCCSNLNYLKYGMLTRKNSKSAMQAGESSSQADAKTEQVEQSMFLQGERTDEKEIPSEQNIVYRGSWYGYIANDKSTSWSGNASNATSGNRAEFTVNFADKKITGTLTADNRQEATFTIDGNIKDNGFEGTAKTAESGFDLDQSNTTRTPKAYITDAKVQGGFYGPKAEELGGWFAYPGDKQTKNATNASGNSSATVVFGAKRQQPVQ

SEQ ID NO: 9 - DNA, 33 bp, Artificial Sequence
  primer
tttcgcgaccacatgaaaagcgtcattccatcc

SEQ ID NO: 10 - DNA, 28 bp, Artificial Sequence
  primer
gttctagagtggcagccctacctctgag

SEQ ID NO: 11 - DNA, 26 bp, Artificial Sequence
  primer
catatggtccctgataaaactgtgag

SEQ ID NO: 12 - DNA, 28 bp, Artificial Sequence
  primer
cgatcgtgaagtttggccaaacatactg

SEQ ID NO: 13 - DNA, 29 bp, Artificial Sequence
  primer
cgatcgaaacacgtatgaaaaatacttag

SEQ ID NO: 14 - DNA, 28 bp, Artificial Sequence
  primer
gttctagagtggcagccctacctctgag

SEQ ID NO: 15 - DNA, 26 bp, Artificial Sequence
  primer
catatggtccctgataaaactgtgag

SEQ ID NO: 16 - DNA, 32 bp, Artificial Sequence
  primer
tctagattaatctgttggggcttctgggcatg

SEQ ID NO: 17 - DNA, 27 bp, Artificial Sequence
  primer
catatggaatgcaagcctgtgaagtgg

SEQ ID NO: 18 - DNA, 28 bp, Artificial Sequence
  primer
gttctagagtggcagccctacctctgag